(19) United States
(12) Patent Application Publication
(10) Pub. No.: US 2025/0230206 A1
(43) Pub. Date: Jul. 17, 2025

(54) SYSTEMS AND METHODS FOR INHIBITING gamma-SECRETASE PRODUCTION OF AMYLOID-beta PEPTIDES

(71) Applicant: Rensselaer Polytechnic Institute, Troy, NY (US)

(72) Inventor: Chunyu Wang, Latham, NY (US)

(73) Assignee: Rensselaer Polytechnic Institute, Troy, NY (US)

(21) Appl. No.: 19/097,101

(22) Filed: Apr. 1, 2025

Related U.S. Application Data
(63) Continuation of application No. 17/352,620, filed on Jun. 21, 2021, now Pat. No. 12,297,244.
Application No. 17/352,620 is a continuation-in-part of application No. PCT/US2020/036244, filed on Jun. 5, 2020.
(60) Provisional application No. 62/857,513, filed on Jun. 5, 2019.
(60) Provisional application No. 63/041,873, filed on Jun. 20, 2020.

(51) Int. Cl. C07K 14/47; A61K 38/00; C12N 9/64

(52) U.S. Cl. CPC C07K 14/4711; C12N 9/6478; A61K 38/00; C07K 2319/03

(57) ABSTRACT

Inhibitors are provided for targeting γ-secretase to reduce amyloid load as a viable strategy in Alzheimer's disease treatment and drug discovery. γ-secretase has been shown to cleave amyloid precursor protein, causing an increase in the extracellular concentration of amyloid-β peptides. This extracellular concentration increase can lead to a build-up of amyloid plaques in patients and associated health complications for them. The inhibitors bind adjacent the transmembrane domain of amyloid precursor protein through both covalent and non-covalent interactions. These interactions inhibit the ability of γ-secretase to cleave the amyloid precursor protein, halting the build-up of extracellular amyloid plaques. The inhibitors exhibit specificity for amyloid precursor proteins, reducing concerns of potential off-target effects.

### CROSS REFERENCE TO RELATED APPLICATION(S)

This application is a continuation of U.S. application Ser. No. 17/352,620, filed Jun. 6, 2021, which is a continuation-in-part of International Application No. PCT/US2020/036244, filed Jun. 5, 2020, which claims the benefit of U.S. Provisional Application No. 62/857,513, filed Jun. 5, 2019, which are incorporated by reference as if disclosed herein in their entireties. This application further claims the benefit of U.S. Provisional Application No. 63/041,873, filed Jun. 20, 2020, which is further incorporated by reference as if disclosed herein in its entirety.

### BACKGROUND

In intramembrane proteolysis (IP), an integral membrane protein is cleaved by intramembrane cleaving proteases (I-CLiPs) within the transmembrane domain (TM) to liberate biologically active fragments. As a unique form of signal transduction, I-CLiPs play essential roles in numerous physiological processes such as embryonic development, immune responses, and normal function of the nervous system.

I-CLiPs also play crucial roles in physiological and pathological processes, such as Alzheimer's disease and cancer. However, the mechanisms of substrate recognition by I-CLiPs remain poorly understood. The aspartic I-CLIP presenilin is the catalytic subunit of the γ-secretase complex, which releases amyloid-β peptides (Aβs) through intramembrane proteolysis of the transmembrane domain of the amyloid precursor protein (APPTM). γ-secretase is a transmembrane protein complex whose catalytic component is the presenilin protein which harbors the active site aspartates. Mutations in presenilin and APP (such as V44M in APPTM) can cause familial AD (FAD) characterized by early onset of dementia and increased Aβ42/Aβ40 ratio.

Unlike soluble proteases which recognize specific amino acid sequences, I-CLiPs display promiscuity against transmembrane substrates. To date, over 90 physiological substrates, e.g., Notch, are known for presenilin/γ-secretase, with no apparent consensus recognition motif. Substrate promiscuity of γ-secretase has contributed to the failure of clinical trials of γ-secretase inhibitors, e.g., through the inhibition of the Notch signaling pathway. Thus understanding substrate/enzyme interaction in I-CLiPs will not only contribute to fundamental understanding of I-CLiPs but also may provide novel insights for selective inhibition of γ-secretase in Alzheimer's disease drug discovery.

Alzheimer's disease (AD) is a progressive neurodegenerative disorder responsible for an increasing number of cases of dementia in the elderly. Without a disease-modifying therapy, there is a pressing need to develop novel and more efficient therapeutic strategies to delay and/or prevent AD occurrence. The neuropathological hallmarks of AD include the presence of senile plaques in the cerebral cortex and hippocampus. These plaques, which are mainly composed of extracellular aggregates of amyloid β-peptides, have been hypothesized to initiate a pathological cascade that results in cognitive decline.

γ-secretase is a promising drug target for reducing amyloid load. γ-Secretase is an aspartyl intramembrane protease, composed of at least four essential proteins, presenilin (PS), nicastrin (Nct), anterior pharynx-defective 1 (Aph-1), and presenilin enhancer 2 (Pen-2), among which PS is the catalytic subunit. Most of the FAD mutations occur within PS genes. Because there are the two isoforms of PS (PS1 and PS2) with distinct substrate specificities and two isoforms of Aph1, there exists at least four major forms of the γ-secretase complexes, with alternative splicing generating even more variant forms of γ-secretase. Thus, γ-secretase is effectively a group of proteases with different subunit compositions and diverse cleavage specificity, as it has been shown that several substrates are cleaved by PS1-containing γ-secretase complex but not by PS2-containing γ-secretase complex. The specific functions of each form of Y-secretase are not known, contributing to the uncertainty in the possible side effects of broad spectrum γ-secretase inhibition. An even more significant obstacle for the development of γ-secretase inhibitors as drug candidates is the large number of substrates of γ-secretase, with over 90 reported to date.

Despite intense effort, clinical trials of anti-Aβ drugs have so far failed. Clinical trials of γ-secretase inhibitors have failed due to serious side effects which may be attributed to the suppression of γ-secretase activity against other substrates, such as Notch, N-cadherin, and tyrosinase. Two broad spectrum γ-secretase inhibitors, avagacestat and semagacestat, failed due to serious adverse effects and worsening cognition in patients. In addition, γ-secretase inhibitors bind directly to PS, the catalytic subunit of γ-secretase. PS is involved in learning and memory, and neuronal survival during aging. PS inhibition by γ-secretase inhibitors has been predicted to result in the accumulation of potentially toxic uncleaved transmembrane substrates of γ-substrate and may have contributed to the declining cognitive ability seen in clinical trials.

Because APPTM is the substrate for γ-secretase to generate Aβ, an alternative and unexplored approach is to target the substrate APPTM instead of γ-secretase to reduce Aβ production. A substrate-specific inhibitor is not expected to affect the γ-secretase cleavage of other physiological substrates, the assembly of the γ-secretase complex, or any presenilin function, thereby sparing the side effects associated with broad-spectrum γ-secretase inhibitors.

Several compounds have been reported to bind to APP and inhibit Aβ production, but it is not clear whether they directly bind to APPTM to exert their effects. Although γ-secretase modulators (GSMs, such as fenofibrate and tarenflurbil) were initially reported to bind to APPTM, no specific binding between GSM and APPTM was found in further studies. The anti-cancer drug bexarotene can reduce amyloid load and alleviate neurodegeneration, but a recent study showed that bexarotene inhibits γ-secretase with low efficacy and this effect is not due to substrate binding.

A major concern for covalent modifiers is its non-specificity and off-target reactivity. Although the pharmaceutical industry traditionally avoids drugs that form covalent bonds with their targets, many first-in-class drugs, such as aspirin, penicillin, and esomeprazole, function through a covalent mechanism and make up about 30% of the drug market. Covalent drugs are indeed a highly effective class of therapeutics with the advantages of high efficacy, due to the zero off-rate. This enables lower dosage and less frequency dosing, which mitigates side effects and off-target effects, and increases compliance. In recent years, covalent compounds have seen a resurgence in drug discovery, with many covalent candidates in drug development pipeline. In particular, covalent compounds can be used to address “undruggable” targets with shallow surfaces and no obvious binding pockets.

### SUMMARY

Some embodiments of the present disclosure are directed to an inhibitor of γ-secretase cleavage of amyloid precursor protein. In some embodiments, the inhibitor includes a structure including:

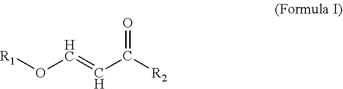

wherein R₁ includes one or more aryl groups, heterocyclic groups, C₇₋₁₀ hydrocarbyl groups, or combinations thereof, and R₂ includes one or more aryl groups, heterocyclic groups, C₇₋₁₀ hydrocarbyl groups, or combinations thereof. In some embodiments, R₁ is substituted with one or more functional groups including hydrocarbyl, halo, halocarbyl, hydroxy, carboxy, alkylamide, cyano, carbonyl, nitro, amino, alkylamino, thiol, mercapto, alkylthio, sulfoxide, sulfone, acyl, acylamino, amidino, aryl, haloaryl, heterocyclic, naphthyl, benzyl, aryloxy, benzyloxy, heteroaryloxy, or benzoyl groups, or combinations thereof. In some embodiments, R₂ is substituted with one or more functional groups including hydrocarbyl, halo, halocarbyl, hydroxy, carboxy, alkylamide, cyano, carbonyl, nitro, amino, alkylamino, thiol, mercapto, alkylthio, sulfoxide, sulfone, acyl, acylamino, amidino, aryl, haloaryl, heterocyclic, naphthyl, benzyl, aryloxy, benzyloxy, heteroaryloxy, or benzoyl groups, or combinations thereof. In some embodiments, the structure includes:

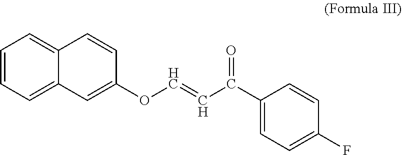 [SMILES: O=C(/C=C/Oc1ccc2ccccc2c1)c1ccc(F)cc1]

Some embodiments of the present disclosure are directed to a method for inhibiting γ-secretase cleavage of amyloid precursor protein including providing a composition including a pharmaceutically effective amount of a structure configured to bind to a domain of an amyloid precursor protein, providing the composition to the domain, and binding the structure to the domain. In some embodiments, the composition includes a pharmaceutically acceptable buffer, diluent, carrier, adjuvant, excipient, or combinations thereof. In some embodiments, the domain includes an amyloid precursor protein transmembrane domain, amyloid precursor protein juxtamembrane region, or combinations thereof. In some embodiments, binding the structure to the domain includes modifying one or more lysine residues of a C-terminal juxtamembrane region adjacent a transmembrane domain. In some embodiments, binding the structure to the domain includes covalent and non-covalent binding. In some embodiments, the concentration of the structure in an environment surrounding the domain after providing the composition is about 25 μM.

In some embodiments of the method, the structure includes:

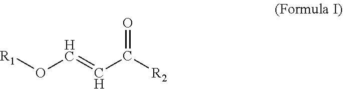

wherein R₁ includes one or more aryl groups, heterocyclic groups, C₇₋₁₀ hydrocarbyl groups, or combinations thereof, and R₂ includes one or more aryl groups, heterocyclic groups, C₇₋₁₀ hydrocarbyl groups, or combinations thereof. In some embodiments, R₁ is substituted with one or more functional groups including hydrocarbyl, halo, halocarbyl, hydroxy, carboxy, alkylamide, cyano, carbonyl, nitro, amino, alkylamino, thiol, mercapto, alkylthio, sulfoxide, sulfone, acyl, acylamino, amidino, aryl, haloaryl, heterocyclic, naphthyl, benzyl, aryloxy, benzyloxy, heteroaryloxy, or benzoyl groups, or combinations thereof. In some embodiments, R₂ is substituted with one or more functional groups including hydrocarbyl, halo, halocarbyl, hydroxy, carboxy, alkylamide, cyano, carbonyl, nitro, amino, alkylamino, thiol, mercapto, alkylthio, sulfoxide, sulfone, acyl, acylamino, amidino, aryl, haloaryl, heterocyclic, naphthyl, benzyl, aryloxy, benzyloxy, heteroaryloxy, or benzoyl groups, or combinations thereof.

Some embodiments of the present disclosure are directed to a method of reducing an amyloid load in a patient to treat a disease including identifying a presence of extracellular aggregates of amyloid-β peptides in the patient, and administering an effective amount of a composition including a structure including:

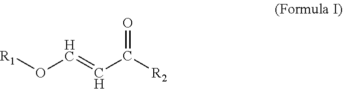

In some embodiments, R₁ includes one or more aryl groups, heterocyclic groups, C₇₋₁₀ hydrocarbyl groups, or combinations thereof, and R₂ includes one or more aryl groups, heterocyclic groups, C₇₋₁₀ hydrocarbyl groups, or combinations thereof. In some embodiments, R₁ is substituted with one or more functional groups including hydrocarbyl, halo, halocarbyl, hydroxy, carboxy, alkylamide, cyano, carbonyl, nitro, amino, alkylamino, thiol, mercapto, alkylthio, sulfoxide, sulfone, acyl, acylamino, amidino, aryl, haloaryl, heterocyclic, naphthyl, benzyl, aryloxy, benzyloxy, heteroaryloxy, or benzoyl groups, or combinations thereof. In some embodiments, R₂ is substituted with one or more functional groups including hydrocarbyl, halo, halocarbyl, hydroxy, carboxy, alkylamide, cyano, carbonyl, nitro, amino, alkylamino, thiol, mercapto, alkylthio, sulfoxide, sulfone, acyl, acylamino, amidino, aryl, haloaryl, heterocyclic, naphthyl, benzyl, aryloxy, benzyloxy, heteroaryloxy, or benzoyl groups, or combinations thereof. In some embodiments, the composition includes a pharmaceutically acceptable buffer, diluent, carrier, adjuvant, excipient, or combinations thereof. In some embodiments, the method includes binding the structure to a domain of an amyloid precursor protein, wherein the domain includes an amyloid precursor protein transmembrane domain, amyloid precursor protein juxtamembrane region, or combinations thereof. In some embodiments, the concentration of the structure in an environment surrounding the domain of amyloid precursor protein after providing the composition is about 25 μM. In some embodiments, the disease is Alzheimer's disease.

In some embodiments of the method, binding the structure to the domain includes modifying one or more lysine residues of a C-terminal juxtamembrane region adjacent a transmembrane domain. In some embodiments, binding the structure to the domain includes covalent and non-covalent binding.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic representation of γ-secretase cleavage that is inhibited by inhibitors according to some embodiments of the present disclosure;

FIG. 2 is a graph portraying the activity of the γ-secretase cleavage inhibitors according to some embodiments of the present disclosure in reducing production of Aβ40 and Aβ42;

FIG. 3 is a chart of a method for inhibiting γ-secretase cleavage of amyloid precursor protein according to some embodiments of the present disclosure;

FIG. 4 is a chart of a method of reducing an amyloid load in a patient to treat a disease according to some embodiments of the present disclosure;

FIGS. 5A-5C is a chart of structures configured to bind to a transmembrane domain of amyloid precursor protein for use in embodiments of the present disclosure;

FIG. 6 is a graph portraying screening results of a DNA-encoded library (DEL) for high affinity binders to amyloid precursor protein;

FIG. 7 portrays chemical structures identified in the DEL screening and graphs showing activity data for those structures;

FIG. 8A portrays a graph showing the inhibitory effects of a structure for use in embodiments of the present disclosure;

FIG. 8B is a graph showing binding of a structure for use in embodiments of the present disclosure at the C-terminal region of amyloid precursor protein; and

FIG. 9 is a graph portraying the activity of the γ-secretase cleavage inhibitors (“C1”) according to some embodiments of the present disclosure in reducing the level of Aβ40, Aβ42 and Aβ42/Aβ40 ratio in HEK 293 cells.

### DESCRIPTION

Some embodiments of the present disclosure are directed to an inhibitor of γ-secretase cleavage of amyloid precursor protein. In some embodiments, the inhibitor includes a structure according to the following Formula I:

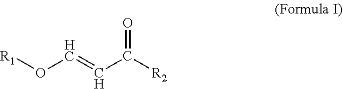

In some embodiments, R₁ includes one or more aryl groups, heterocyclic groups, C₇₋₁₀ hydrocarbyl groups, or combinations thereof. In some embodiments, R₂ includes one or more aryl groups, heterocyclic groups, C₇₋₁₀ hydrocarbyl groups, or combinations thereof. As used herein, the term “hydrocarbyl” is used to refer to branched linear, unbranched linear, and cyclic structures of carbon and hydrogen atoms, including saturated, unsaturated, substituted, and unsubstituted forms, as well as forms including one or more of N, O, or S, or combinations thereof. In some embodiments, R₁ is substituted with one or more functional groups including hydrocarbyl, halo, halocarbyl, hydroxy, carboxy, alkylamide, cyano, carbonyl, nitro, amino, alkylamino, thiol, mercapto, alkylthio, sulfoxide, sulfone, acyl, acylamino, amidino, aryl, haloaryl, heterocyclic, naphthyl, benzyl, aryloxy, benzyloxy, heteroaryloxy, or benzoyl groups, or combinations thereof. In some embodiments, R₂ is substituted with one or more functional groups including hydrocarbyl, halo, halocarbyl, hydroxy, carboxy, alkylamide, cyano, carbonyl, nitro, amino, alkylamino, thiol, mercapto, alkylthio, sulfoxide, sulfone, acyl, acylamino, amidino, aryl, haloaryl, heterocyclic, naphthyl, benzyl, aryloxy, benzyloxy, heteroaryloxy, or benzoyl groups, or combinations thereof. In some embodiments, R₁ and R₂ are separated by a linker region. In some embodiments, the linker region includes an unsaturated ketone.

In some embodiments, the inhibitor is configured to bind, modify, or bind and modify amyloid precursor protein. In some embodiments, the inhibitor binds/modifies amyloid precursor protein covalently, non-covalently, or combinations thereof. In some embodiments, the inhibitor binds/modifies amyloid precursor protein at a transmembrane domain thereof. In some embodiments, the inhibitor binds/modifies amyloid precursor protein adjacent the transmembrane domain, e.g., a C-terminal juxtamembrane region. In some embodiments, the inhibitor binds/modifies amyloid precursor protein at one or more lysine residues at or adjacent the transmembrane domain, as will be discussed in greater detail below.

In some embodiments, the inhibitor includes a structure according to the following Formula II:

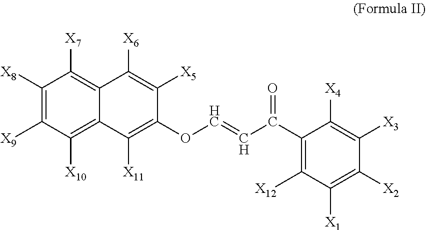

In some embodiments, each of X₁-X₁₂ is one of a hydrogen, hydrocarbyl, halo, halocarbyl, hydroxy, carboxy, alkylamide, cyano, carbonyl, nitro, amino, alkylamino, thiol, mercapto, alkylthio, sulfoxide, sulfone, acyl, acylamino, amidino, aryl, haloaryl, heterocyclic, naphthyl, benzyl, aryloxy, benzyloxy, heteroaryloxy, or benzoyl group, or combinations thereof. In an exemplary embodiment, the structure includes the following Formula III:

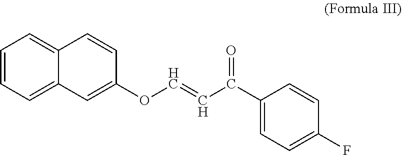 [SMILES: O=C(/C=C/Oc1ccc2ccccc2c1)c1ccc(F)cc1]

In an exemplary embodiment, the structure includes the following Formula IV:

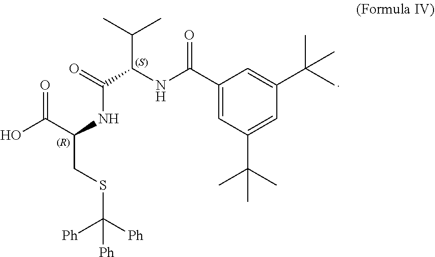

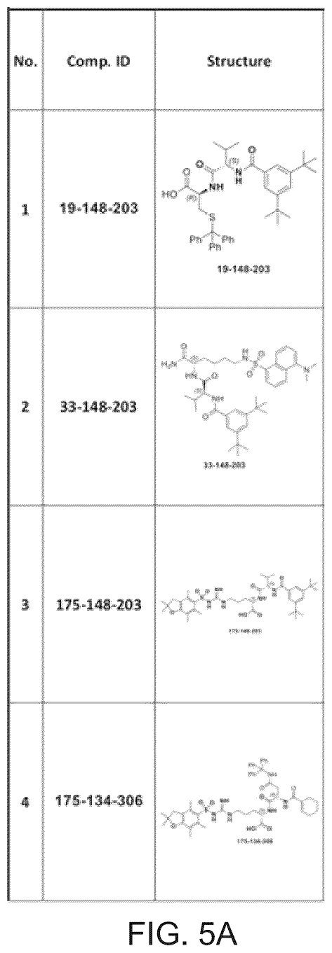

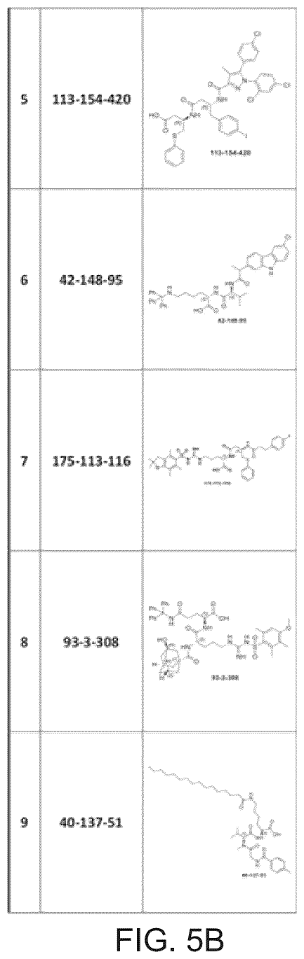

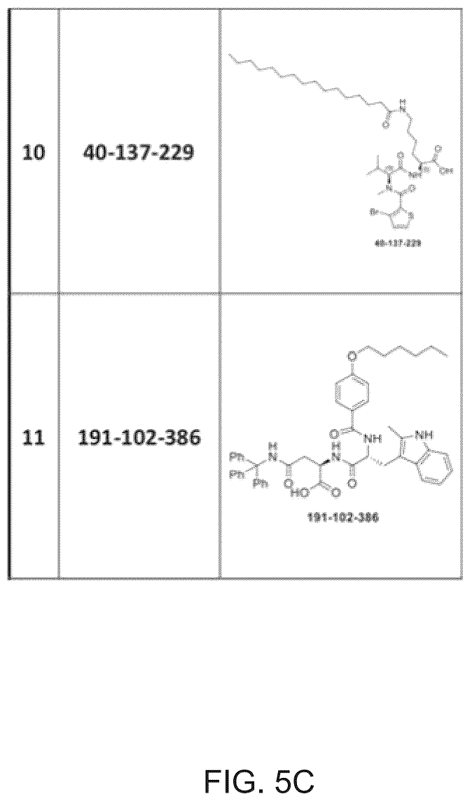

In some embodiments, the structure includes at least one of the structures identified at FIGS. 5A-5C. Referring specifically to FIG. 5C, in some embodiments, the carboxylic acid group is changed to one or more acetyl groups, e.g., to include a negative charge. In some embodiments, the chiral center is changed. In some embodiments, the aryl substitutions are changed, e.g., as removing/replacing the isobutyl group with fluorine, chlorine, —OCH₃, etc., or combinations thereof. In some embodiments, the tri-aryl group, the sulfur, or combinations thereof are changed.

In some embodiments, the inhibitor is incorporated into a composition for use, e.g., in vitro, in vivo, etc. In some embodiments, the inhibitor is incorporated into a composition for use as a pharmaceutical or nutraceutical. In some embodiments, the composition includes a pharmaceutically acceptable buffer, diluent, carrier, adjuvant, excipient, etc., or combinations thereof.

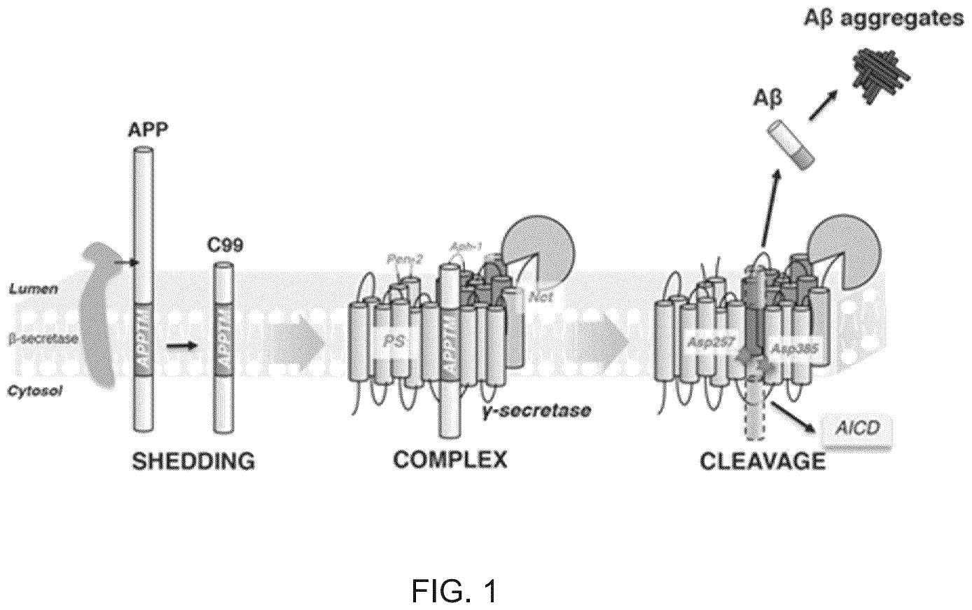

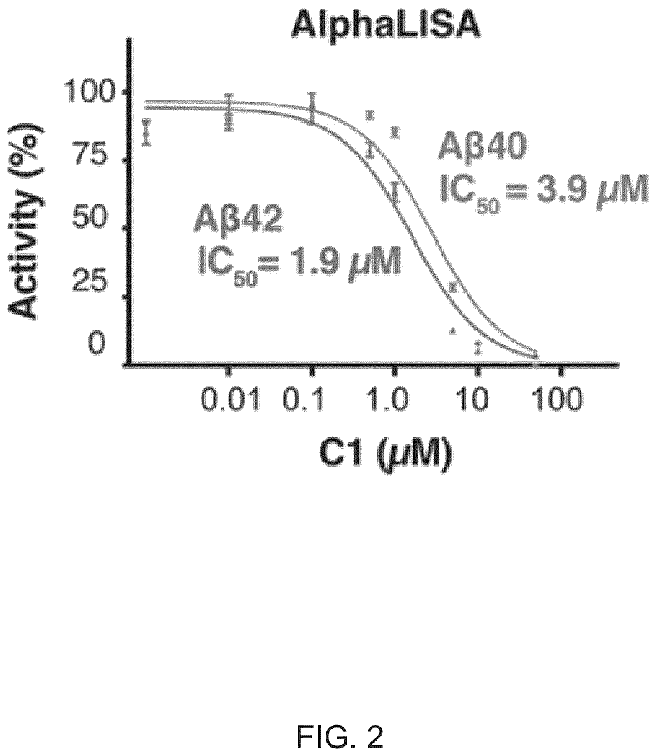

Referring now to FIG. 1, and without wishing to be bound by theory, γ-secretase cleaves within the transmembrane domain of the amyloid precursor protein (APPTM) to release AB, which aggregates to form neurotoxic oligomers and fibrils. To demonstrate that interaction of the inhibitory structures according to embodiments of the present disclosure and APP substrate can inhibit γ-secretase cleavage thereof, AlphaLISA assays were employed with γ-secretase in the HeLa membrane and biotinylated Sb4 based on the sequence of APP as the substrate. Formula III decreased the production of Aβ40 and Aβ42 by γ-secretase in a dose dependent manner (FIG. 2). An IC₅₀ value of 1.9 μM was obtained for the inhibition of Aβ42 production and 3.9 μM for the inhibition of Aβ40 production. The lower IC₅₀ for Aβ42 production compared to Aβ40 indicated that Formula III has selectivity in inhibiting Aβ42 production over Aβ40. Similar inhibition effects were also observed in a gel-based assay using MBP-APPTM fusion protein as the substrate and the presenilin homolog (PSH) MAMRE50 as an enzyme.

Without wishing to be bound by theory, chemical structures consistent with the present disclosure covalently modify APPTM. The α,β-unsaturated ketone in the structure is a Michael's acceptor for covalent modification of proteins. The electro-deficient β-carbon reacts with nucleophiles in proteins, such as the amino group in the side chain of lysine residues. APP includes a C-terminal juxtamembrane lysine cluster (K53, K54 and K55). Juxtamembrane residues of APP have been shown by mutagenesis to play an important role in γ-secretase cleavage, most likely through interaction with transmembrane loops in γ-secretase. The C-terminal juxtamembrane lysine cluster of APPTM is near the ε-cleavage sites T48 and L49, where initial cleavage of the protein occurs. C-terminal α-helix in APPTM unwinds into an extended β-conformation to expose the ε-cleavage sites, forming an intermolecular β-sheet with two β-strands. Modification of the C-terminal juxtamembrane lysines of APPTM interferes with the α to β conformational transition and/or the formation of the intermolecular β-sheet, inhibiting γ-secretase cleavage.

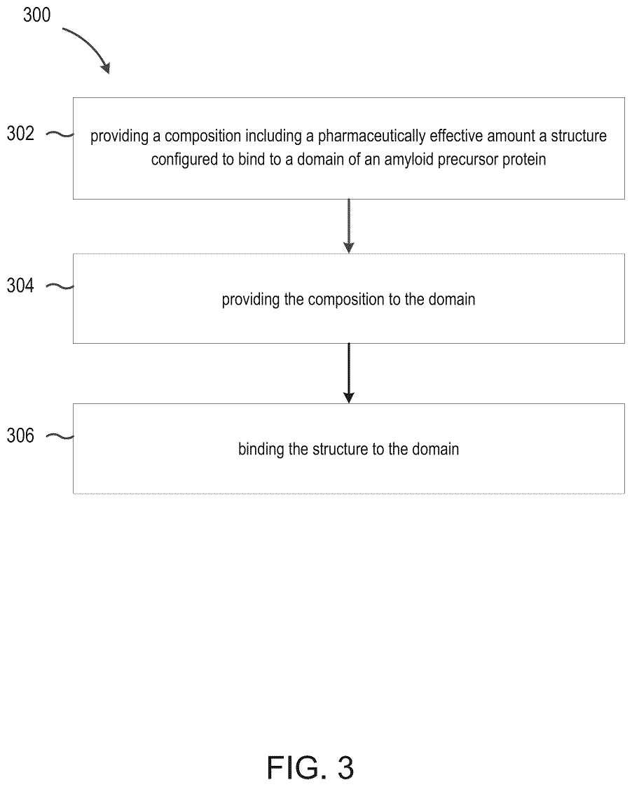

Referring now to FIG. 3, in some embodiments, the present disclosure is directed to a method 300 for inhibiting γ-secretase cleavage of amyloid precursor protein. At 302, a composition is provided. As discussed above, the composition includes a structure configured to bind to a domain of amyloid precursor protein. In some embodiments, the composition includes a pharmaceutically effective amount of the structure. In some embodiments, the pharmaceutically effective amount is sufficient to produce a localized concentration of structure of about 10 μM, 15 μM, 20 μM, or 25 μM, as will be discussed in greater detail below. In some embodiments, the composition includes a pharmaceutically acceptable buffer, diluent, carrier, adjuvant, excipient, or combinations thereof.

As discussed above, in some embodiments, the structure includes:

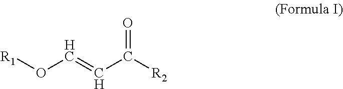

In some embodiments, R₁ includes one or more aryl groups, heterocyclic groups, C₇₋₁₀ hydrocarbyl groups, or combinations thereof. In some embodiments, R₂ includes one or more aryl groups, heterocyclic groups, C₇₋₁₀ hydrocarbyl groups, or combinations thereof. In some embodiments, R₁ is substituted with one or more functional groups including hydrocarbyl, halo, halocarbyl, hydroxy, carboxy, alkylamide, cyano, carbonyl, nitro, amino, alkylamino, thiol, mercapto, alkylthio, sulfoxide, sulfone, acyl, acylamino, amidino, aryl, haloaryl, heterocyclic, naphthyl, benzyl, aryloxy, benzyloxy, heteroaryloxy, or benzoyl groups, or combinations thereof. In some embodiments, R₂ is substituted with one or more functional groups including hydrocarbyl, halo, halocarbyl, hydroxy, carboxy, alkylamide, cyano, carbonyl, nitro, amino, alkylamino, thiol, mercapto, alkylthio, sulfoxide, sulfone, acyl, acylamino, amidino, aryl, haloaryl, heterocyclic, naphthyl, benzyl, aryloxy, benzyloxy, heteroaryloxy, or benzoyl groups, or combinations thereof. In some embodiments, R₁ and R₂ are separated by a linker region. In some embodiments, the linker region includes an unsaturated ketone.

In some embodiments, the composition includes the structure according to the following Formula II:

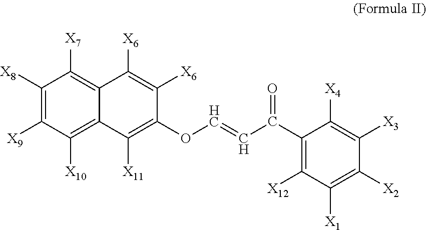

In some embodiments, each of X₁-X₁₂ is one of a hydrogen, hydrocarbyl, halo, halocarbyl, hydroxy, carboxy, alkylamide, cyano, carbonyl, nitro, amino, alkylamino, thiol, mercapto, alkylthio, sulfoxide, sulfone, acyl, acylamino, amidino, aryl, haloaryl, heterocyclic, naphthyl, benzyl, aryloxy, benzyloxy, heteroaryloxy, or benzoyl group, or combinations thereof. In an exemplary embodiment, the structure includes the following Formula III:

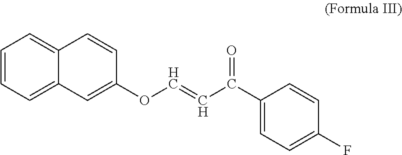 [SMILES: O=C(/C=C/Oc1ccc2ccccc2c1)c1ccc(F)cc1]

In an exemplary embodiment, the structure includes the following Formula IV:

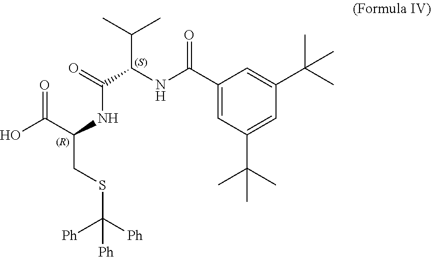

At 304, the composition is provided to amyloid precursor protein. At 306, the structure is bound to the amyloid precursor protein. In some embodiments, the structure binds/modifies amyloid precursor protein covalently, non-covalently, or combinations thereof. In some embodiments, the structure binds/modifies amyloid precursor protein at a transmembrane domain thereof, adjacent the transmembrane domain, e.g., a C-terminal juxtamembrane region, or combinations thereof. In some embodiments, the inhibitor binds/modifies amyloid precursor protein at one or more lysine residues at or adjacent the transmembrane domain, e.g., at one or more lysine residues of a C-terminal juxtamembrane region adjacent a transmembrane domain. In some embodiments, the concentration of the structure in an environment surrounding the domain after providing the composition is about 10 μM, 15 μM, 20 μM, or 25 μM.

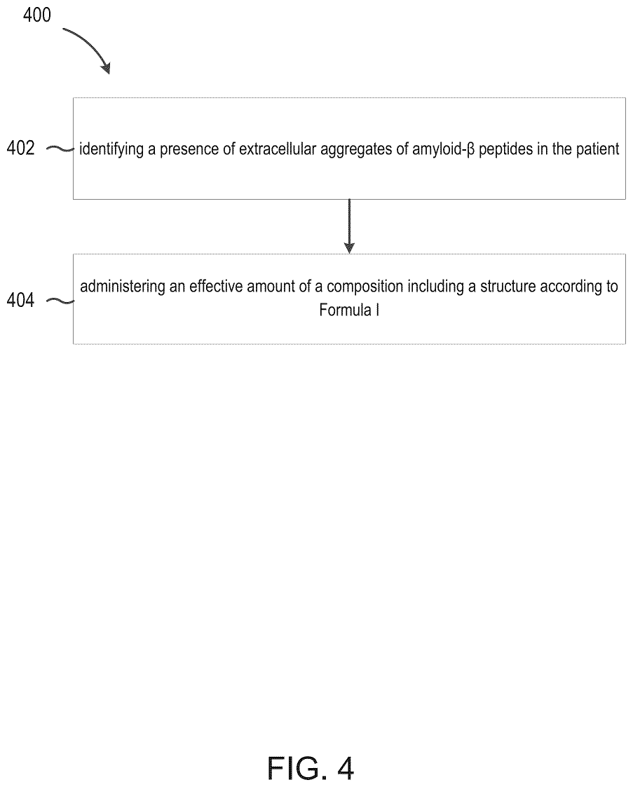

Referring now to FIG. 4, some embodiments of the present disclosure are directed to a method 400 of reducing an amyloid load in a patient to treat a disease. In some embodiments, the disease being treated is one resulting from or causing or believed to be resulting from or causing the buildup of amyloid plaques within the patient. In some embodiments, the disease includes Alzheimer's disease.

At 402, a presence of extracellular aggregates of amyloid-β peptides is identified in the patient. The identification of extracellular aggregates of amyloid-β peptides in the patient is performed via any suitable method, such as testing of a sample from the patient, identification of aggregates in situ, etc. At 404, a pharmaceutically effective amount of a composition including a structure configured to bind to a domain of amyloid precursor protein is administered. In some embodiments, the pharmaceutically effective amount is sufficient to produce a localized concentration of structure of about 10 μM, 15 μM, 20 μM, or 25 μM. In some embodiments, the composition includes a pharmaceutically acceptable buffer, diluent, carrier, adjuvant, excipient, or combinations thereof.

As discussed above, in some embodiments, the structure includes:

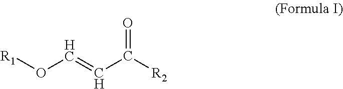

In some embodiments, R₁ includes one or more aryl groups, heterocyclic groups, C₇₋₁₀ hydrocarbyl groups, or combinations thereof. In some embodiments, R₂ includes one or more aryl groups, heterocyclic groups, C₇₋₁₀ hydrocarbyl groups, or combinations thereof. In some embodiments, R₁ is substituted with one or more functional groups including hydrocarbyl, halo, halocarbyl, hydroxy, carboxy, alkylamide, cyano, carbonyl, nitro, amino, alkylamino, thiol, mercapto, alkylthio, sulfoxide, sulfone, acyl, acylamino, amidino, aryl, haloaryl, heterocyclic, naphthyl, benzyl, aryloxy, benzyloxy, heteroaryloxy, or benzoyl groups, or combinations thereof. In some embodiments, R₂ is substituted with one or more functional groups including hydrocarbyl, halo, halocarbyl, hydroxy, carboxy, alkylamide, cyano, carbonyl, nitro, amino, alkylamino, thiol, mercapto, alkylthio, sulfoxide, sulfone, acyl, acylamino, amidino, aryl, haloaryl, heterocyclic, naphthyl, benzyl, aryloxy, benzyloxy, heteroaryloxy, or benzoyl groups, or combinations thereof. In some embodiments, R₁ and R₂ are separated by a linker region. In some embodiments, the linker region includes an unsaturated ketone.

In some embodiments, the composition includes the structure according to the following formula II:

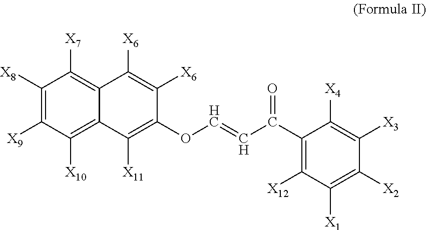

In some embodiments, each of X₁-X₁₂ is one of a hydrogen, hydrocarbyl, halo, halocarbyl, hydroxy, carboxy, alkylamide, cyano, carbonyl, nitro, amino, alkylamino, thiol, mercapto, alkylthio, sulfoxide, sulfone, acyl, acylamino, amidino, aryl, haloaryl, heterocyclic, naphthyl, benzyl, aryloxy, benzyloxy, heteroaryloxy, or benzoyl group, or combinations thereof. In an exemplary embodiment, the structure includes the following Formula III:

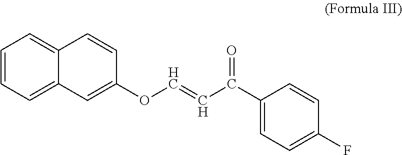 [SMILES: O=C(/C=C/Oc1ccc2ccccc2c1)c1ccc(F)cc1]

In an exemplary embodiment, the structure includes the following Formula IV:

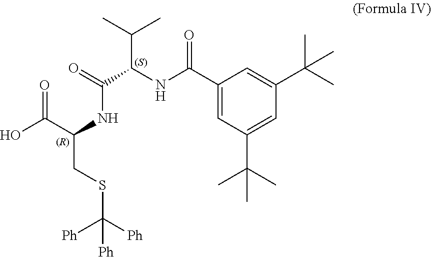

In some embodiments, the structure binds/modifies amyloid precursor protein covalently, non-covalently, or combinations thereof. In some embodiments, the structure binds/modifies amyloid precursor protein at a transmembrane domain thereof, adjacent the transmembrane domain, e.g., a C-terminal juxtamembrane region, or combinations thereof. In some embodiments, the structure binds/modifies amyloid precursor protein at one or more lysine residues at or adjacent the transmembrane domain, e.g., at one or more lysine residues of a C-terminal juxtamembrane region adjacent a transmembrane domain. As discussed above, modification of the C-terminal juxtamembrane lysines of APPTM interferes with the α to β conformational transition and/or the formation of the intermolecular β-sheet, inhibiting γ-secretase cleavage. In turn, cleavage inhibition reduces the production of Aβ40 and Aβ42, which have been shown to have a central role in the conditions including Alzheimer's disease, Down's syndrome, toxicity from Aβ aggregates, neuron inflammation, potentiation of tau pathology, etc. In some embodiments, the concentration of the structure in an environment surrounding the domain after providing the composition is about 10 μM, 15 μM, 20 μM, or 25 μM.

### Methods

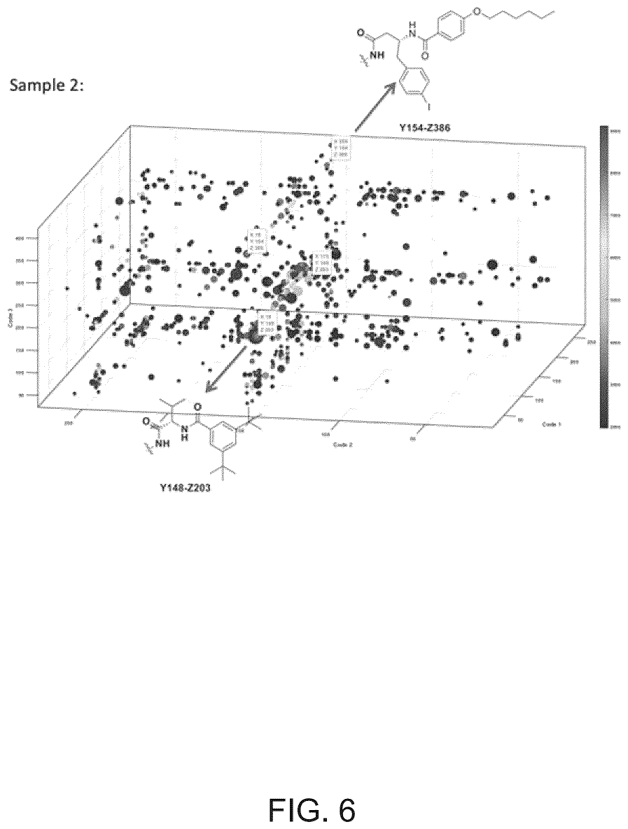

Referring now to FIG. 6, a tripeptide DNA-encoded library (DEL) composed of ˜20 million compounds was screened. A binding assay was carried out using Nickel-beads immobilized APPTM and DEL, PCR amplification and deep DNA sequencing. Highly-enriched compounds were then synthesized and characterized. DEL data are represented in scatter plot format. Each point represents a unique compound, with x, y, z coordinates providing the identity of the compound. The size the of the point represents fold enrichment of the compound from DEL screening compared with the initial DEL, which is a measure of the affinity between the compound and the target protein APPTM, while the color of the point encodes the number of reads in DNA deep sequencing.

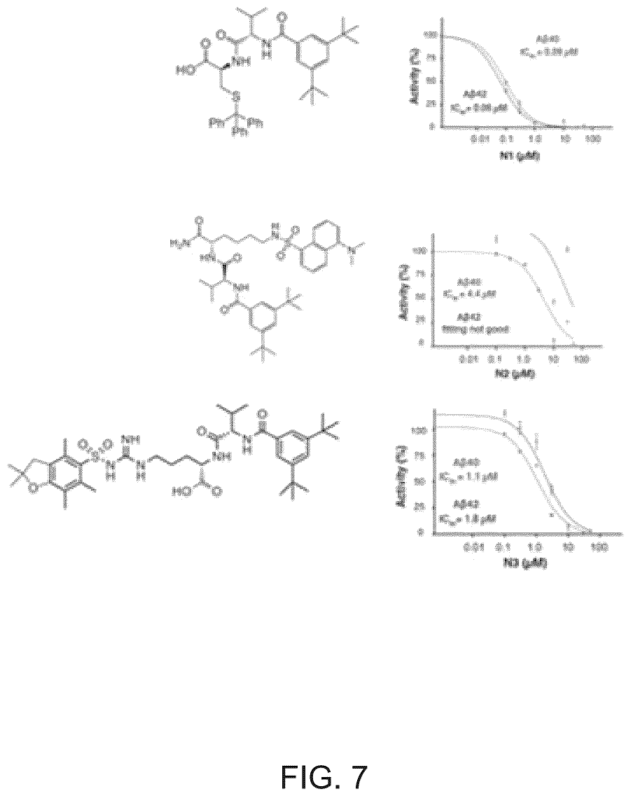

Eleven compounds were synthesized in a simple, gel-based intramembrane proteolysis assay and then chose three most active compounds for AlphaLISA γ-secretase assay. These three compounds from DEL screening all showed efficacy in γ-secretase assay to various degrees (see FIG. 7), with N1 being the most potent with IC50˜0.1 μM.

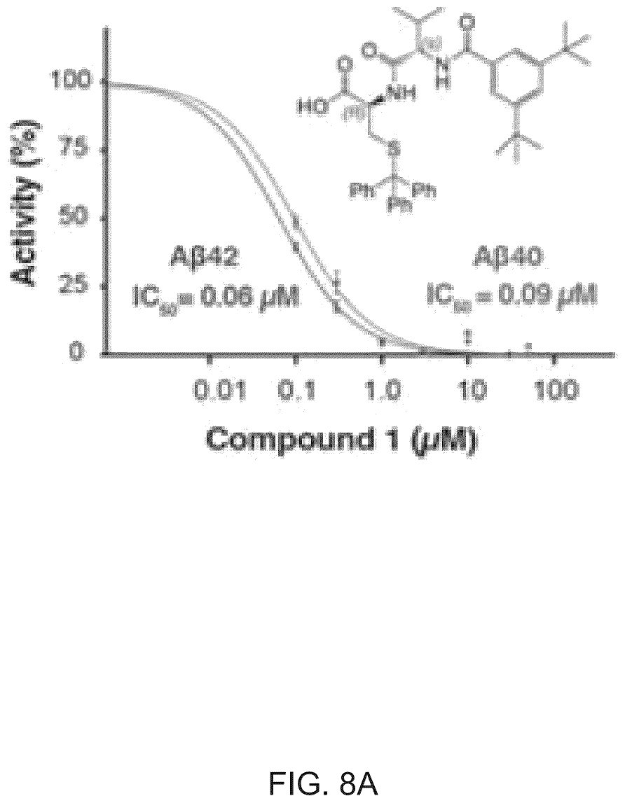

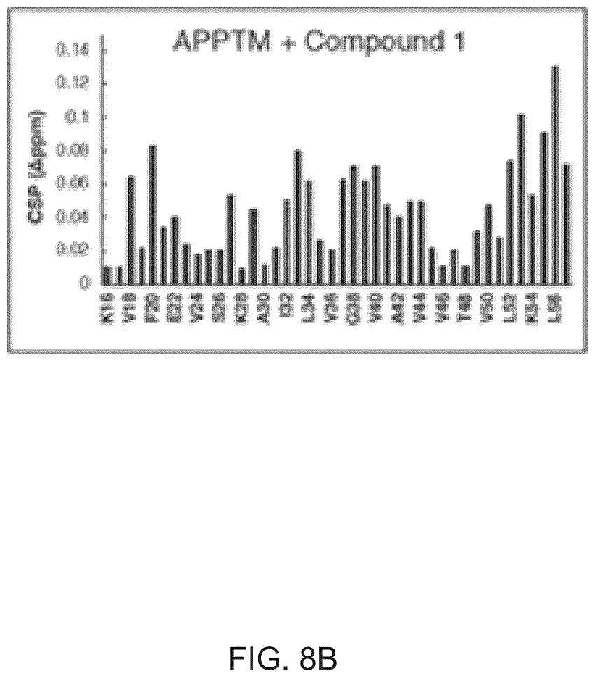

Referring now to FIGS. 8A-8B, N1 was further characterized by NMR titration and mass spectroscopy and was found to bind non-covalently to C-terminal region of APPTM, mostly likely through electrostatic interaction with the C-terminal lysine cluster of APPTM. Compound N1 inhibits A production in AlphaLISA-secretase assay (see FIG. 8A). Further, N1 binds APPTM at the C-terminal region, non-covalently in NMR titration (see FIG. 8B).

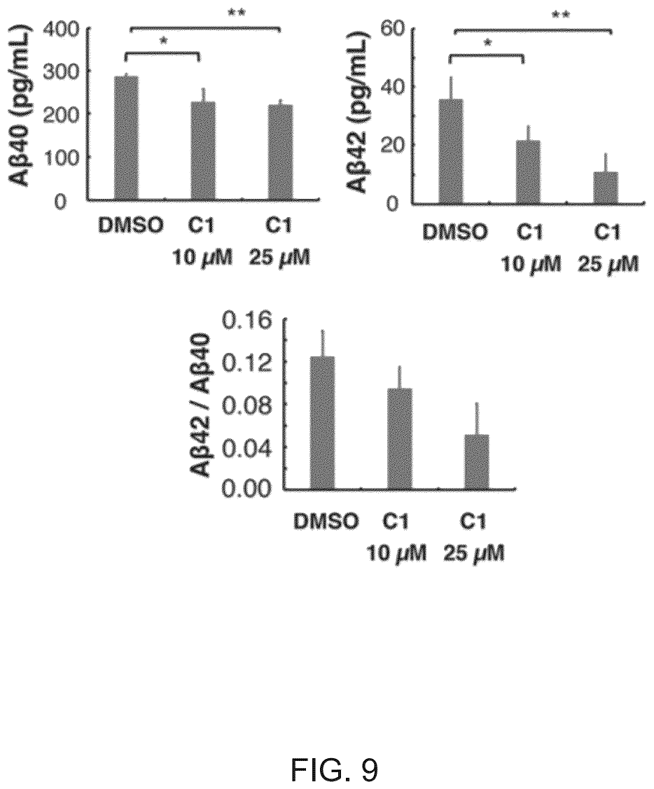

The effect of the structures on Aβ40 and Aβ42 production by γ-secretase was tested in human embryonic kidney 293 (HEK 293) cells using a sandwich ELISA assay. HEK 293 cells were transfected with a plasmid to express human APP695, and Aβ40 and Aβ42 levels were measured in the conditioned medium. After the treatment by 10 μM and 25 μM of inhibitor for 24 h, the amount of Aβ40 decreased by ˜25% (FIG. 9). In contrast, Aβ42 decreased by ˜30% after treatment by 10 UM of the structure for 24 h, and by ˜70% after treatment by 25 μM of the structure. In agreement with the AlphaLISA data, the inhibitor reduces the Aβ42 level more than that of Aβ40 in a cellular environment, reducing the Aβ42/Aβ40 ratio.

Methods and system of the present disclosure are advantageous to inhibit γ-secretase cleavage as evidenced by AlphaLISA assay, with an IC₅₀ of 1.9 μM for Aβ42 and 3.9 UM for Aβ40. This suggests that targeting the substrate of γ-secretase to reduce amyloid load is a viable strategy in Alzheimer's disease drug discovery. Further, the compositions of the present disclosure selectively modify APPTM in the presence of ubiquitin, showing specificity for APPTM, most likely due to its non-covalent interaction with APPTM and high reactivity of K55.

Without wishing to be bound by theory, targeting the substrate of γ-secretase alone can be sufficient for reducing Aβ production. Targeting Aβ precursor substrates of γ-secretase offers a direction in Alzheimer's disease drug discovery for reducing amyloid load as disease-modifying therapy. Furthermore, the methodology may be adapted to additional γ-secretase substrates thus enabling assessment of functional consequences of the γ-secretase cleavage of specific substrates. Further, the method inhibits Aβ production without affecting the γ-secretase activity against other non-APP substrates or presenilin function.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A method for inhibiting γ-secretase cleavage of amyloid precursor protein, comprising:
providing a composition including a pharmaceutically effective amount of an inhibitor having a structure configured to bind to a transmembrane domain of an amyloid precursor protein;
providing the inhibitor to the domain to form an inhibitor complex with the domain;
allowing the inhibitor complex to interact with γ-secretase, so that cleavage of the amyloid precursor protein by γ-secretase is inhibited, wherein the binding of the structure to the domain is selected from the group consisting of covalent binding, non-covalent binding, and combinations thereof.

2. The method according to claim 1, wherein the inhibitor includes at least one amide group, and at least one aromatic ring.

3. The method according to claim 1, wherein the concentration of the inhibitor in an environment surrounding the domain after providing the composition is about 25 μM.

4. The method according to claim 1, wherein the inhibitor structure is selected from the group consisting of:

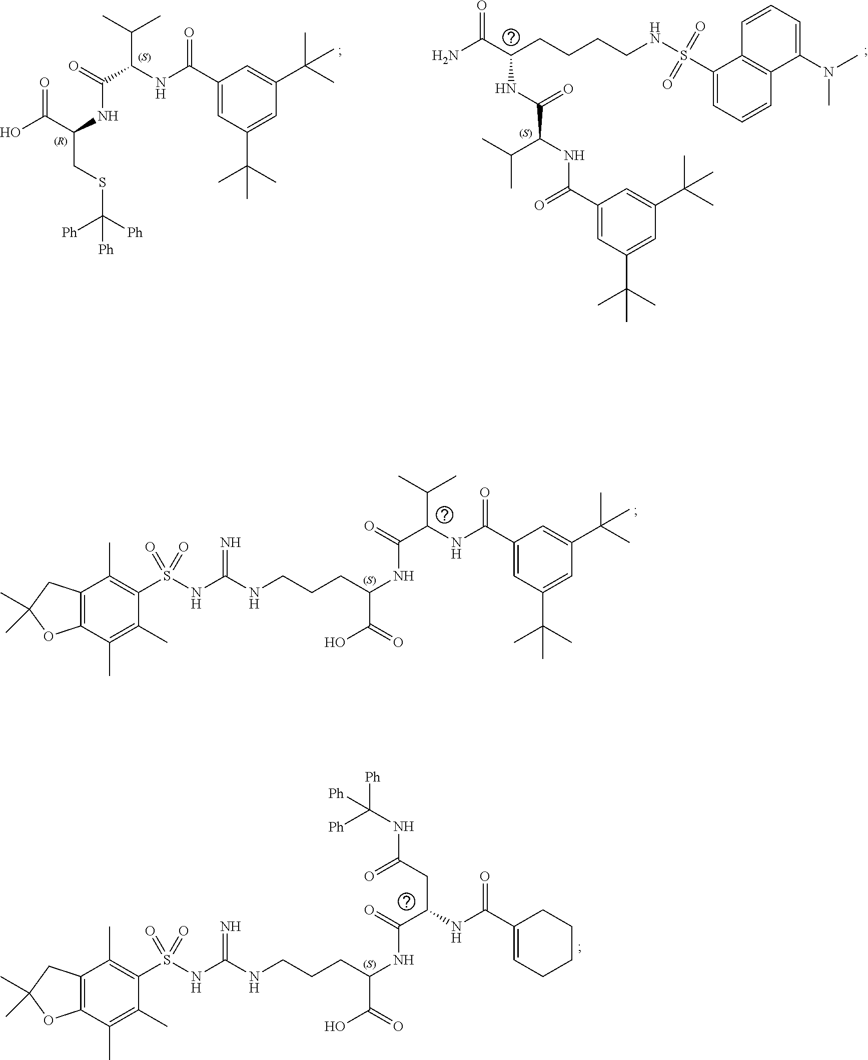

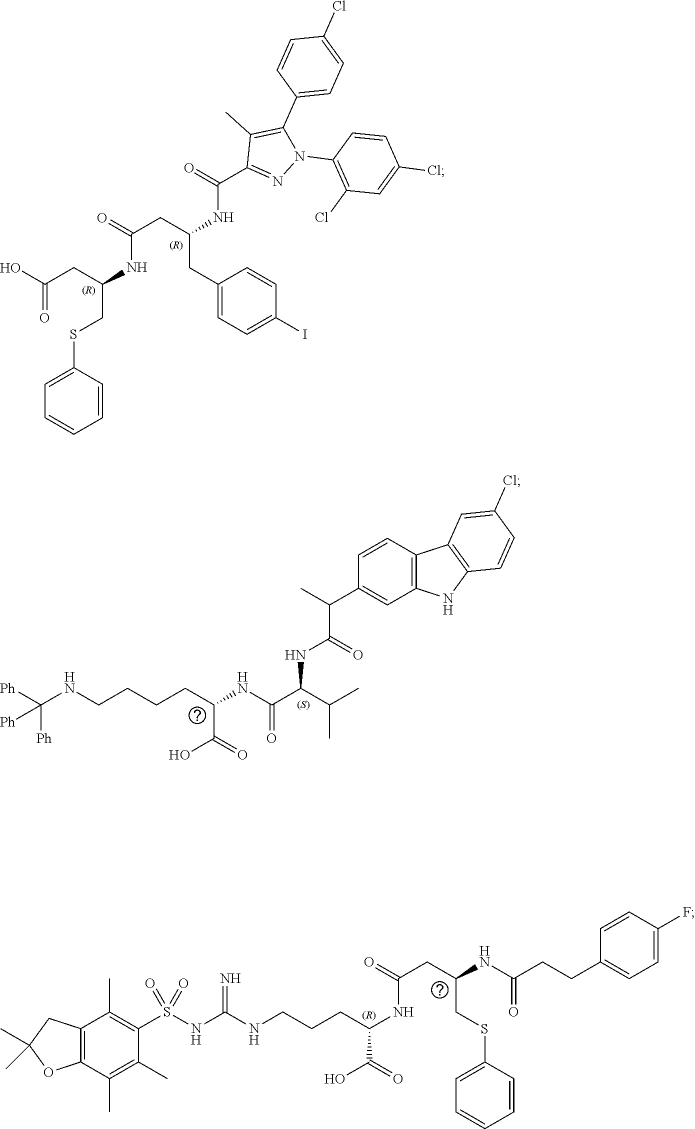

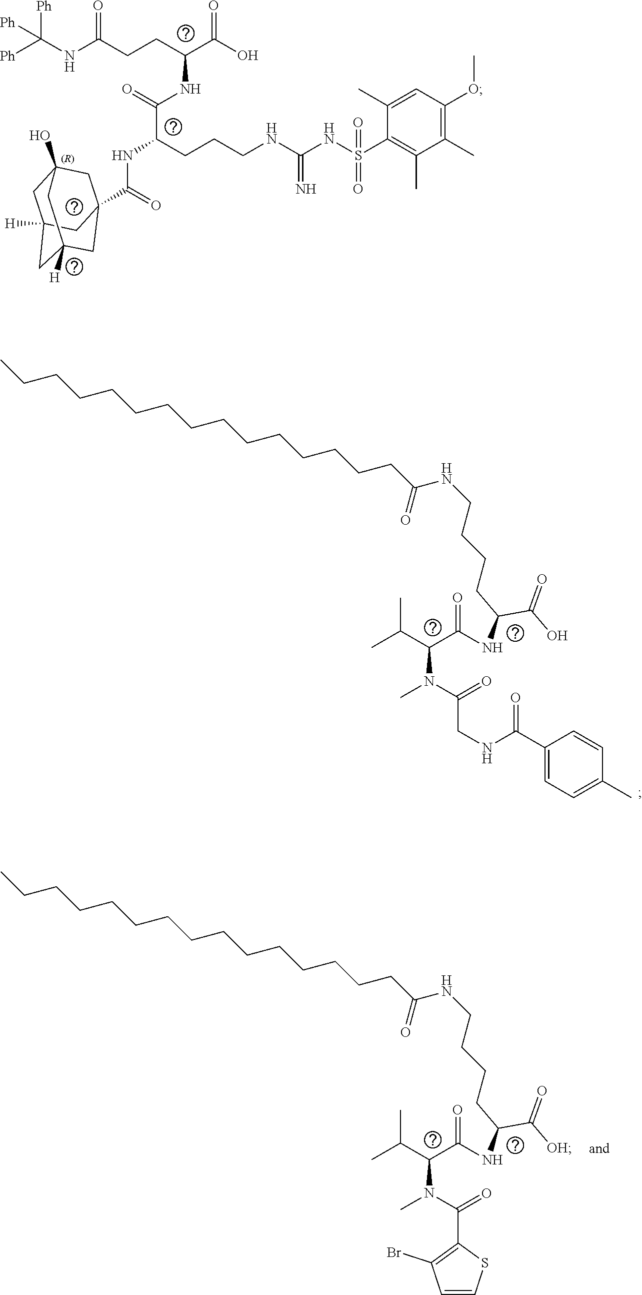

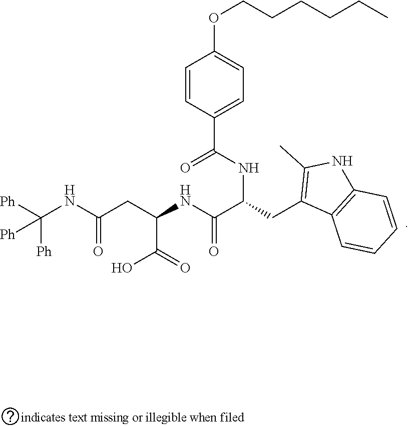 [SMILES: CCCCCCOc1ccc(C(=O)N[C@H](Cc2c(C)[nH]c3ccccc23)C(=O)N[C@H](CC(=O)NC(c2ccccc2)(c2ccccc2)c2ccccc2)C(=O)O)cc1]

5. The method according to claim 1, wherein the inhibitor structure includes: 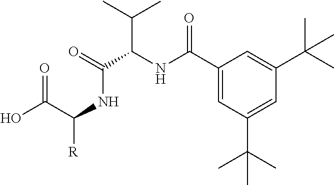
wherein R is substituted with one or more functional groups selected from the group consisting of: aryl groups, heterocyclic groups, hydrocarbyl groups, and combinations thereof.

6. The method according to claim 1, wherein the inhibitor structure includes:

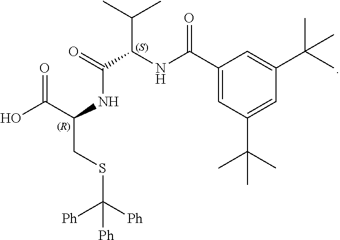

7. An inhibitor of γ-secretase cleavage of amyloid precursor protein, comprising:
a structure configured to bind to a transmembrane domain of an amyloid precursor protein to form an inhibitor complex with the domain, so that cleavage of the amyloid precursor protein by γ-secretase is inhibited,
wherein the binding of the structure to the domain is selected from the group consisting of covalent binding, non-covalent binding, and combinations thereof.

8. The inhibitor according to claim 7, wherein the structure is chosen from the group consisting of:

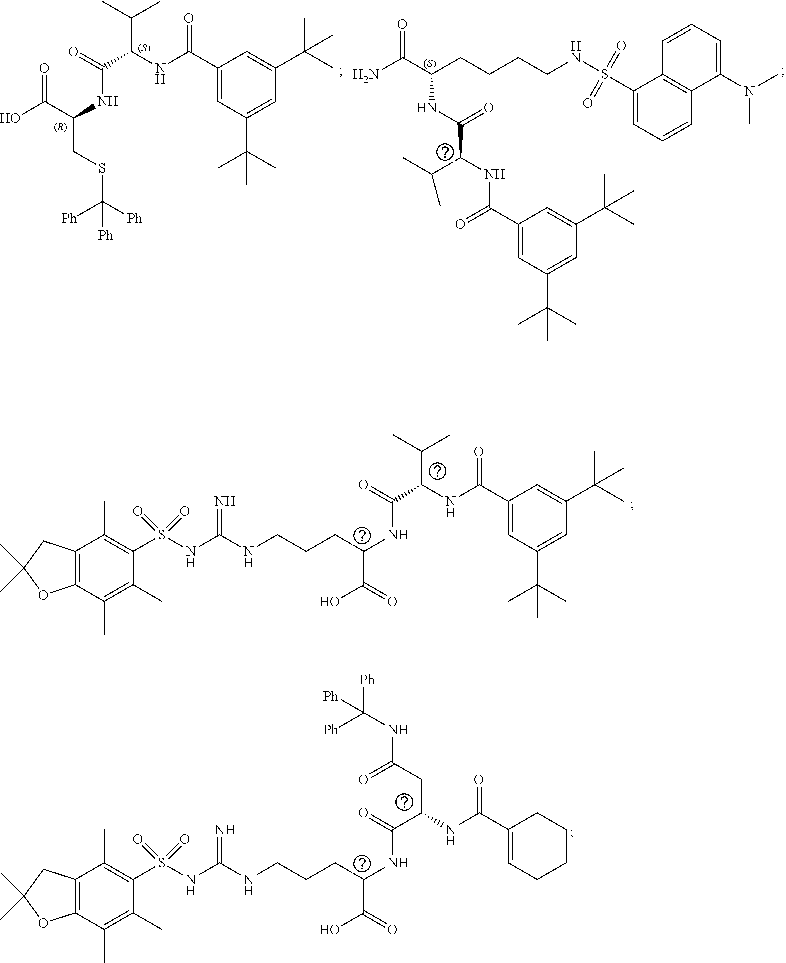

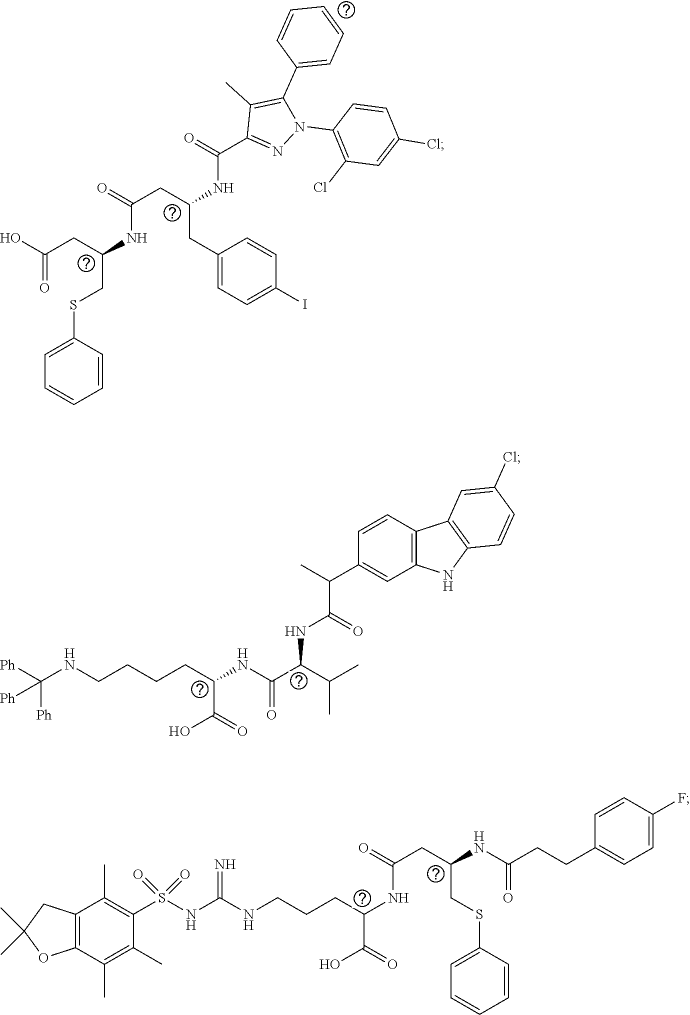 [SMILES: Cc1c(C(=O)N[C@@H](CC(=O)N[C@@H](CSc2ccccc2)CC(=O)O)Cc2ccc(I)cc2)nn(-c2ccc(Cl)cc2Cl)c1-c1ccccc1 ; CC(C(=O)N[C@H](C(=O)N[C@@H](CCCCNC(c1ccccc1)(c1ccccc1)c1ccccc1)C(=O)O)C(C)C)c1ccc2c(c1)[nH]c1ccc(Cl)cc12 ; Cc1c(C)c(S(=O)(=O)NC(=N)NCCCC(NC(=O)C[C@H](CSc2ccccc2)NC(=O)CCc2ccc(F)cc2)C(=O)O)c(C)c2c1OC(C)(C)C2]

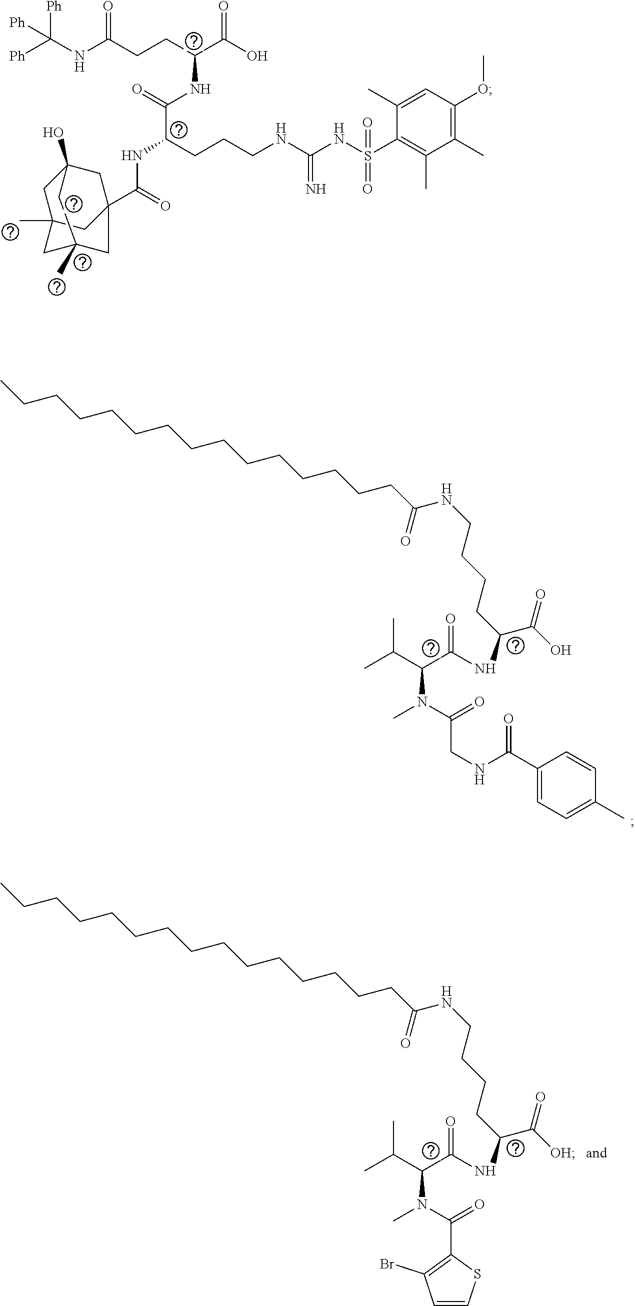

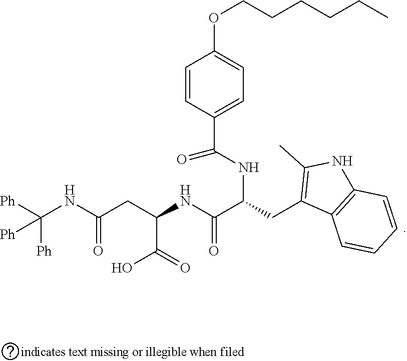 [SMILES: CCCCCCOc1ccc(C(=O)N[C@H](Cc2c(C)[nH]c3ccccc23)C(=O)N[C@H](CC(=O)NC(c2ccccc2)(c2ccccc2)c2ccccc2)C(=O)O)cc1]

9. The inhibitor according to claim 7, wherein the inhibitor structure includes: 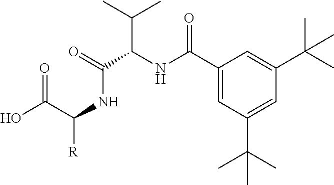
wherein R is substituted with one or more functional groups selected from the group consisting of: aryl groups, heterocyclic groups, hydrocarbyl groups, and combinations thereof.

10. The inhibitor according to claim 7, wherein the inhibitor structure includes:

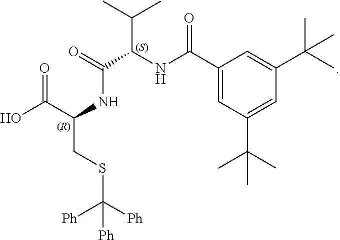

11. A method of reducing an amyloid load in a patient to treat a disease, comprising:
identifying a presence of extracellular aggregates of amyloid-β peptides in the patient; and administering an effective amount of a composition including an inhibitor according to claim 8.

12. The method according to claim 11, wherein the composition includes a pharmaceutically acceptable additive selected from the group consisting of a buffer, a diluent, a carrier, an adjuvant, an excipient, and combinations thereof.

13. The method according to claim 11, further comprising:
binding the inhibitor to a domain of an amyloid precursor protein,
wherein the domain includes an amyloid precursor protein transmembrane domain, amloid precursor protein juxtamembrane region, or combinations thereof.

14. The method according to claim 13, wherein the binding of the structure to the domain further comprises:
modifying one or more lysine residues of a C-terminal juxtamembrane region adjacent a transmembrane domain.

15. The method according to claim 11, wherein the concentration of the structure in an environment surrounding the domain of amyloid precursor protein after providing the composition is about 25 μM.

16. The method according to claim 11, wherein the disease is Alzheimer's disease.

17. The method according to claim 11, wherein the structure includes:

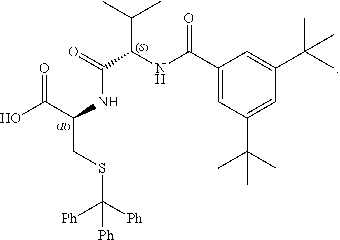

18. A method for inhibiting γ-secretase cleavage of amyloid precursor protein, comprising:
providing a composition including a pharmaceutically effective amount of a structure configured to bind to a domain of an amyloid precursor protein,
providing the composition to the domain; and
binding the structure to the domain,
wherein the structure includes: 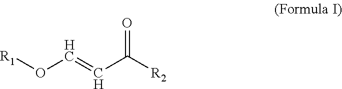
wherein R₁ includes one or more aryl groups, heterocyclic groups, C₇₋₁₀ hydrocarbyl groups, or combinations thereof, and R₂ includes one or more aryl groups, heterocyclic groups, C₇₋₁₀ hydrocarbyl groups, or combinations thereof,
wherein binding the structure to the domain further comprises:
modifying one or more lysine residues of a C-terminal juxtamembrane region adjacent a transmembrane domain.
